(19) United States
(12) Patent Application Publication
(10) Pub. No.: US 2023/0285177 A1
(43) Pub. Date: Sep. 14, 2023

(54) BODILY WASTE COLLECTION DEVICES, SYSTEMS, AND METHODS

(71) Applicant: PureWick Corporation, Covington, GA (US)

(72) Inventors: Jill W. Jones, Avondale Estates, GA (US); Michael W. Drobnik, Downers Grove, IL (US); Christopher D. Drobnik, Wauconda, IL (US); Patrick Hudson Chancy, Dunwoody, GA (US); Hoang D. Nguyen, Smyrna, GA (US)

(21) Appl. No.: 17/999,648

(22) PCT Filed: Jun. 1, 2021
(86) PCT No.: PCT/US2021/035181

§ 371 Date: Nov. 22, 2022

Related U.S. Application Data
(60) Provisional application No. 63/033,310, filed on Jun. 2, 2020.

(51) Int. Cl. A61F 5/44; A61F 5/443

(52) U.S. Cl. CPC A61F 5/4404; A61F 5/443

(57) ABSTRACT

Examples relate to devices, systems, and methods of using non-intrusive bodily waste collection devices. The devices, systems, and methods of disclosed herein include a non-intrusive bodily waste collection device having an outermost fluid impermeable barrier forming a chamber therein, a filter bag disposed in the chamber, an annular body defining an opening into the chamber and that is sized and shaped to interface with a wearer's anus or stoma, and one or more ports on the fluid impermeable barrier for removing fluids therefrom.

### CROSS-REFERENCE TO RELATED APPLICATIONS

This application claims priority to U.S. Provisional Patent Application No. 63/033,310, filed 2 Jun. 2020, the disclosure of which is incorporated herein, in its entirety, by this reference.

### BACKGROUND

An individual may have limited or impaired mobility such that bowel movements in a restroom are challenging or impossible. For example, the individual may have a condition, had a surgery, or a have disability that impairs mobility. In another example, the individual may have restricted travel conditions such as those experience by pilots, drivers, and workers in hazardous areas. Additionally, stool collection from the individual may be needed for monitoring purposes or clinical testing.

Bed pans and ostomy bags, may be used to address some of these circumstances. However, bed pans and ostomy bags have several problems associated therewith. For example, bed pans may be prone to discomfort, spills, and other hygiene issues. Ostomy bags require intrusive surgery. For example, using ostomy bags requires a surgical procedure to make an ostomy and stoma to connect to the ostomy bag, which may be painful, prone to infections, and leave permanent scarring or other side effects.

Thus, users and manufacturers of stool collection devices continue to seek new and improved devices, systems, and methods to collect stool.

### SUMMARY

Embodiments disclosed herein are related to devices, systems, and methods of using non-intrusive bodily waste collection devices. In an embodiment, a bodily waste collection device is disclosed. The bodily waste collection device includes an annular body defining an opening therein, the annular body being configured to be positioned over an anus or stoma of a wearer to position the opening around the anus or stoma. The bodily waste collection device includes a fluid impermeable barrier affixed to the annular body, the fluid impermeable barrier having an outer surface and an inner surface, the inner surface at least partially defining a chamber within the fluid impermeable barrier. The bodily waste collection device includes a filter bag disposed within the chamber and positioned to receive waste via the opening. The bodily waste collection device includes one or more ports disposed on the fluid impermeable barrier and configured to attach to a drainage tube.

In an embodiment, a bodily waste collection system is disclosed. The bodily waste collection system includes a fluid storage container configured to hold a fluid. The bodily waste collection system includes a bodily waste collection device fluidly coupled to the fluid storage container, the bodily waste collection device including an annular body defining an opening therein, the annular body being configured to be positioned over an anus or a stoma of a wearer to position the opening around the anus or the stoma; a fluid impermeable barrier affixed to the annular body, the fluid impermeable barrier having an outer surface and an inner surface, the inner surface at least partially defining a chamber within the fluid impermeable barrier; a filter bag disposed within the chamber and positioned to receive waste via the opening; and one or more ports disposed on the fluid impermeable barrier and configured to attached to a drainage tube. The bodily waste collection system further includes a drainage tube in fluid communication with the one or more ports. The bodily waste collection system includes a vacuum source fluidly coupled to one or more of the fluid storage container or the bodily waste collection device via the drainage tube, the vacuum source configured to draw fluid from the chamber via the drainage tube.

In an embodiment, a method to collect bodily waste is disclosed. The method includes positioning an opening of a bodily waste collection device over an anus or a stoma of a wearer, the bodily waste collection device including: an annular body defining an opening therein, the annular body being configured to be positioned over the anus or the stoma of the wearer to position the opening around the anus or stoma; a fluid impermeable barrier affixed to the annular body, the fluid impermeable barrier having an outer surface and an inner surface, the inner surface at least partially defining a chamber within the fluid impermeable barrier; a filter bag disposed within the chamber and positioned to receive waste via the opening; and one or more ports disposed on the fluid impermeable barrier and configured to attached to a drainage tube. The method includes receiving waste from the anus or stoma into the filter bag in the chamber. The method includes filtering fluid from the waste with the filter bag.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is an isometric view of a bodily waste collection device, according to an embodiment.

FIGS. 2 and 3 are isometric views of the annular body of FIG. 1 in various conformational states, according to embodiments.

FIG. 4 is a top plan view of the annular body of FIG. 1, according to an embodiment.

FIG. 5 is an isometric view of the underside of the annular body having the filter bag bonded thereto, according to an embodiment.

FIG. 6 is an isometric view of the underside of the annular body having the filter bag bonded thereto, according to an embodiment.

FIG. 7 is an isometric view of a partial assembly of a bodily waste collection device, according to an embodiment.

FIG. 8 is an isometric view of a bodily waste collection device, according to an embodiment.

FIG. 9 is an isometric view of a bodily waste collection device, according to an embodiment.

FIG. 10 is a schematic of a bodily waste collection system, according to an embodiment.

FIG. 11 is a schematic of a bodily waste collection system during use, according to an embodiment.

FIG. 12 is a flow diagram of a method to collect bodily waste, according to an embodiment.

### DETAILED DESCRIPTION

Embodiments disclosed herein are related to devices, systems, and methods of using bodily waste collection devices. The devices, systems, and methods of disclosed herein include a non-intrusive bodily waste collection device having an outermost fluid impermeable barrier forming a chamber therein, a filter bag disposed in the chamber, an annular body defining an opening into the chamber and that is sized and shaped to interface with a wearer's anus or stoma, and one or more ports on the fluid impermeable barrier for removing fluids therefrom. The waste is received into filter bag via the opening. Gravity and/or suction pull fluid from the waste which passes through the filter bag while the solids of the waste are retained in the filter bag. The fluid moves to a gravimetrically low portion or lowest pressure portion of the chamber. The fluid is removed via one or more ports fluidly coupled with one or more of a fluid storage container or vacuum.

By filtering the fluid from the waste and retaining the solids in the filter bag, the devices, systems, and methods disclosed herein preventing clogging of drainage tubes, allow longer use of waste collection devices by reducing the volume of waste therein, and allow waste content monitoring. Additionally, attaching the bodily waste collection device to the patient with the annular body prevents spillage of waste during bowel movement or excretion. The bodily waste collection devices are particularly effective for collecting semi-solid stool.

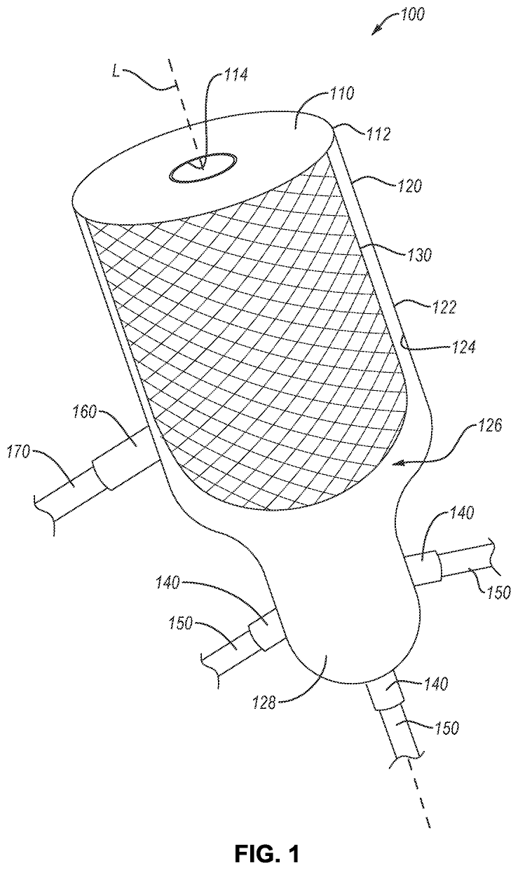

FIG. 1 is an isometric view of a bodily waste collection device 100, according to an embodiment. The bodily waste collection device 100 includes an annular body 110, a fluid impermeable barrier 120 forming a chamber 126 therein, a filter bag 130 disposed within the chamber 126, and one or more ports 140 disposed on the fluid impermeable barrier 120. The device 100 is configured to be attached to a wearer to collect the waste from the wearer and remove liquids from the waste. The annular body 110 is attached to the wearer around the wearer's anus or stoma to position the device 100 for use. Waste enters the chamber 126 via an opening 114 in the annular body 110, the filter bag 130 in the chamber 126 filters the solids from the waste and allows the fluids to pass therethrough, the liquid moves to a gravimetrically low point of the chamber 126 and is removed therefrom, such as via vacuum.

The annular body 110 includes a thin interfacing layer 112 having the opening 114 therein. The annular body 110 may be a complete or partial annulus. For example, the annular body 110 may be a disc-shaped body with a hole therein, such as an annular plate. In some examples, the annular body 110 may include a flat sheet of material with a hole located at or near an edge of the material to form a partial annulus. The interfacing layer 112 is fluid impermeable. For example, the interfacing layer may include a polymer, silicone, or the like. The interfacing layer 112 may be constructed of a smooth and/or soft material such as a closed cell foam, silicon, a smooth plastic, or may include an outwardly facing layer of relatively smooth material and/or soft material (e.g., silicone). The interfacing layer 112 may be deformable responsive to bending forces. For example, the annular body 110 may be compliant for folding, bending, or the like. The interfacing layer 112 may be an annular disc, sheet, plate, or other body having the opening 114 within an outermost periphery thereof. The interfacing layer 112 is sized and shaped to fit within the intergluteal cleft of a wearer, such as when bent, folded or otherwise manipulated.

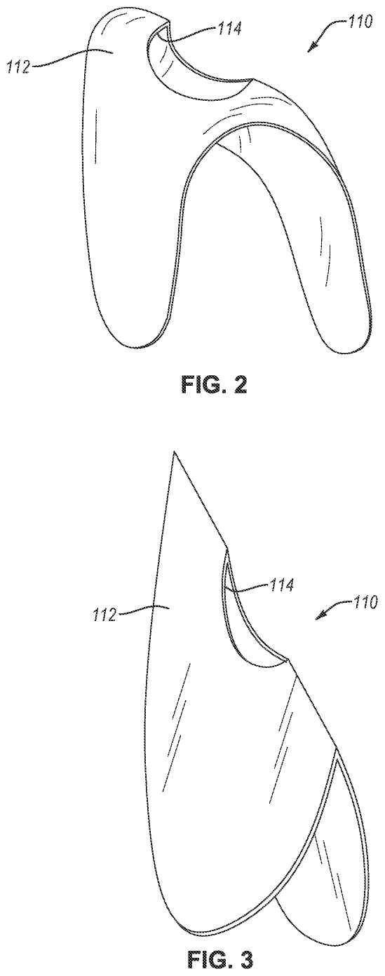

The interfacing layer 112 may be deformed to complement the anatomy of a wearer, such as to fit within the intergluteal cleft. FIGS. 2 and 3 are isometric views of the annular body 110 in various conformational states, according to embodiments. As shown in FIG. 2, the annular body 110 (interfacing layer 112) may be bendable to comply to pressure applied thereto, such as from a wearer or medical professional. The annular body 110 may be composed of a material and have a thickness that allows the annular body 110 to bend responsive to external forces, without breaking.

As shown in FIG. 3, the annular body 110 (interfacing layer 112) may be foldable to comply to pressure applied thereto, such as from a wearer or medical professional. The annular body 110 may be composed of a material and have a thickness that allows the annular body to fold responsive to external forces, without breaking. For example, a medical professional or a wearer may fold and/or bend the annular body 110 in half to insert the annular body between the buttocks of the wearer to be disposed within the intergluteal fold during use. Such a fold may form a cusp, having at least a portion of the opening 114 therein. Accordingly, the annular body 110 may be provided as a generally planar body, a bent body, or a folded body. By folding or bending the annular body 110, a wearer or user (e.g., medical professional) may non-intrusively position the opening 114 at least partially around the anus or a stoma of a wearer.

Returning to FIG. 1, the opening 114 may be disposed within the outer periphery of the interfacing layer 112. As shown, the opening 114 may be disposed at or near a centroid of the interfacing layer 112. The opening 114 may be disposed closer to the outer periphery of the interfacing layer 112 than illustrated. The opening 114 is sized and shaped to fit around (e.g., over) the anus or stoma of the patient without occluding the stoma or anus. For example, the opening 114 may be circular, elliptical, rectangular, or any other shape. Accordingly, the annular body 110 is sized and shaped to be positioned over an anus or stoma of a wearer to position the opening 114 around the anus or stoma. Waste may be passed into the bodily waste collection device 100 via the opening 114.

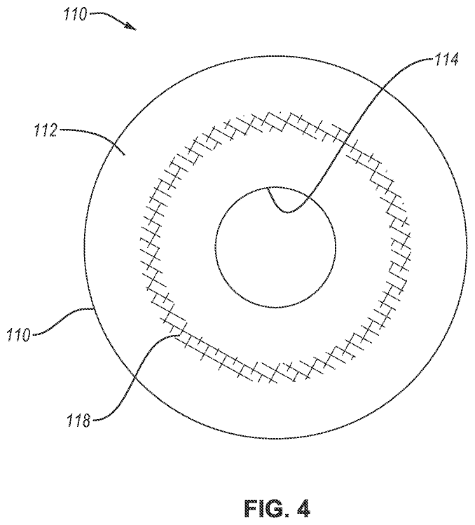

FIG. 4 is a top plan view of the annular body 110 of FIG. 1, according to an embodiment. As shown in FIG. 4, the interfacing layer 112 of the annular body 110 may include an adhesive 118 thereon to adhere to the skin of a subject, such as within the intergluteal cleft. The adhesive 118 may be integrally formed in or disposed on an outward facing surface of the interfacing layer 112. Such adhesive 118 may secure the bodily waste collection device 100 in position such that the opening 114 is maintained around or at least proximate to the anus or stoma of the wearer. The adhesive 118 may be disposed at one or more points between the opening 114 and the outer periphery of the annular body 110. The adhesive 118 may include a medical adhesive or any other adhesive that is safe to use against the skin of humans, such as acrylate adhesives (e.g., methacrylates, epoxy diacrylates, or cyanoacrylate adhesives), silicone adhesives, hydrogels, or the like.

While shown as a substantially continuous ring on the interfacing layer 112, the adhesive 118 may be disposed on the interfacing layer 112 as one or more spots, dots, or strips. The adhesive 118 may be disposed on the interfacing layer 112 as one or more of rings of adhesive extending around the opening 114. In some examples (not shown), substantially the entire interfacing layer 112 may be covered by adhesive 118.

Returning to FIG. 1, the fluid impermeable barrier 120 is attached to the annular body 110 to at least temporarily retain received bodily waste therein and prevent leakage to the outside environment. The fluid impermeable barrier 120 includes an open mouth at a first (e.g., upper) end and a closed second (e.g., lower) end. The fluid impermeable barrier 120 is formed from a fluid impermeable material, such as portions (e.g., layers) including a polymer, a metal film, rubber, or the like. For example, the fluid impermeable barrier 120 may include silicone, polypropylenes, polyethylenes, polyethylene terephthalates, polystyrenes, polyurethanes, polycarbonates, polyamides, polyesters, polyacrylates, polychloroprene, vinyl, polyvinyl chloride, poly(vinyl imidazole), latex, silanes (e.g., an halogenated alkyl silane), perfluorinated polymers, polytetrafluoroethylene (PTFE), chlorosulphonate polyolefins, polyethylene oxide, blends or copolymers of any of the foregoing, or any other fluid impermeable polymer.

The fluid impermeable barrier 120 may be transparent in one or more portions thereof. The fluid impermeable barrier 120 may be translucent or opaque in one or more portions thereof. For example, the fluid impermeable barrier 120 may include a transparent window around the filter bag or in a lower portion of the fluid impermeable barrier 120 to allow a user or wearer to monitor waste and liquids therein.

The fluid impermeable barrier 120 includes an outer surface 122 and an inner surface 124. The outer surface 122 may be relatively smooth or soft to prevent sores or discomfort for the wearer. The inner surface 124 defines a chamber 126. The chamber 126 receives and retains the bodily waste therein. Liquid from the bodily waste may travel through the chamber 126 from the upper region of the chamber 126 to a lower region (e.g., gravimetrically low point) of the chamber 126.

The mouth of the fluid impermeable barrier 120 is mated to the annular body 110, such as at the outer periphery of the annular body 110. Accordingly, waste received into the bodily waste collection device 100 enters the chamber 126 via the opening 114 of the annular body 110 mated to the fluid impermeable barrier 120. The mouth of the fluid impermeable barrier 120 may be mated to the annular body 110 at the outer periphery thereof, at one or more points internal to the outer periphery such as at the inner periphery of the annular body 110 (e.g., opening 114), or any points between the outer periphery and the opening 114.

The mouth of the fluid impermeable barrier 120 may be adhered, welded, or otherwise affixed to the annular body 110. At least a portion of the fluid impermeable barrier 120 (e.g., mouth of the fluid impermeable barrier 120) may be integrally formed with the annular body 110, such as having a one-piece construction. In such examples, one or more portions of the fluid impermeable barrier may be open as initially formed, and later sealed after the filter bag is installed.

The fluid impermeable barrier 120 may have a substantially cylindrical or conical shape. For example, the fluid impermeable barrier 120 may include an upper portion having a greater outer dimension than a lower portion of the fluid impermeable barrier 120. The fluid impermeable barrier 120 may be shaped as a pouch having opposing sides that are joined along the edges thereof. In some examples, the fluid impermeable barrier 120 may include a sump 128 in a portion of the fluid impermeable barrier 120 intended to be positioned at the gravimetrically low portion of device 100 during use. The sump 128 may include a portion of the fluid impermeable barrier 120 sized and shaped to direct the fluid in the chamber to one or more ports 140 disposed on the fluid impermeable barrier 120 in the sump 128. For example, the sump 128 may include a narrower portion of the chamber 126 than an upper portion. The sump 128 may be a side channel or pocket in the chamber 126, a conical shaped terminus of the chamber 126, or the like.

The bodily waste collection device 100 includes the filter bag 130 disposed within the chamber 126, and positioned to receive waste via the opening 114 of the annular body 110. The filter bag 130 may have a length that is shorter than a length of the fluid impermeable barrier 120. Accordingly, the bottom of the filter bag 130 does not extend to the bottom of the fluid impermeable barrier 120. The filter bag 130 may be substantially cylindrical with a closed bottom substantially opposite to the mouth of the filter bag 130. In some examples, the filter bag is conically shaped. In some examples, the filter bag is configured as or shaped like a pouch.

The filter bag 130 may include a bag or pouch constructed of a mesh material (e.g., mesh bag). The mesh material has a sieve size selected to allow fluids to pass therethrough and retain solids therein. The sieve size (e.g., mesh size) may include at least about A five micrometer (μm) mesh, such as about 5 μm to about 1,500 μm, about 100 μm to about 1000 μm, about 250 μm to about 500 μm, about 500 μm to about 1,000 μm, less than about 1,500 μm, less than about 1,000 μm, less than about 800 μm, less than about 400 μm, or larger than about 100 μm. The filter bag 130 may be seamless. The filter bag 130 may include one or more layers of mesh material, such as a single layer or dual layers.

The mesh material may include fibers of a polymer or natural material. For example, the mesh material may include fibers of a polymer, such as nylon, polypropylenes, polyethylenes, polyethylene terephthalates, polystyrenes, polyurethanes, polycarbonates, polyamides, polyesters, polyacrylates, polychloroprene, vinyl, polyvinyl chloride, poly(vinyl imidazole), latex, silanes (e.g., an halogenated alkyl silane), perfluorinated polymers, polytetrafluoroethylene (PTFE), chlorosulphonate polyolefins, polyethylene oxide, blends or copolymers of any of the foregoing. The mesh may be formed of natural fibers such as cotton (e.g., cheesecloth). The mesh material may be woven or non-woven. The mesh material may be a felt of any of the materials disclosed above.

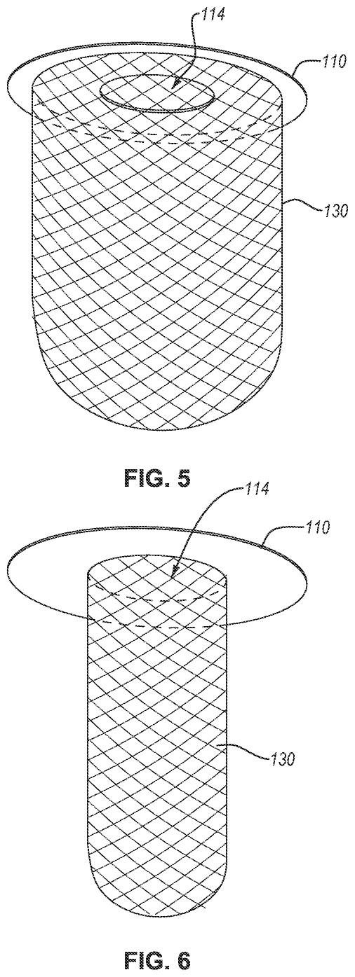

The filter bag 130 may be affixed the annular body 110 or the fluid impermeable barrier 120. For example, the filter bag 130 may be affixed to the annular body 110 between the opening 114 and the outer periphery thereof. FIG. 5 is an isometric view of the underside of the annular body 110 having the filter bag 130 bonded thereto, according to an embodiment. As shown, the filter bag 130 may be affixed to the underside of the annular body 110 at points between the outer periphery of the annular body 110 and the opening 114. In such examples, the filter bag 130 is spaced from the fluid impermeable barrier 120. By spacing the filter bag 130 from the fluid impermeable barrier 120, a space is created between the filter bag and the fluid impermeable barrier to allow the liquid to separate from the solids in the bodily waste, no matter the orientation of the wearer. A mouth of the filter bag 130 may be bound to the underside of the annular body 110 such that the opening 114 is contained within the filter bag 130. The mouth of the filter bag 130 may be bound to the annular body 110 to encircle the opening 114. Put another way, waste passed into the opening 114 may be disposed within the filter bag 130 first. The mouth of the filter bag 130 may be bound to the underside of the annular body 110 by adhesive (e.g., any adhesive), stitching, heat welding, staples, integral formation, or the like.

The filter bag 130 may be affixed to the annular body 110 at least proximate to the opening 114. FIG. 6 is an isometric view of the underside of the annular body 110 having the filter bag 130 bonded thereto, according to an embodiment. As shown in FIG. 6, the filter bag 130 is affixed to the annular body 110 at the opening 114. For example, the filter bag 130 may be adhered, sewn, stapled, or otherwise attached to the annular body at the opening 114.

The filter bag 130 may be affixed to the annular body 110 at an outer periphery of the annular body 110. In some examples (not shown), the filter bag 130 is affixed to the top of the annular body 110 (e.g., at the outwardly facing portion of the interfacing layer 112), such as by extending out of the chamber 126 from the opening 114.

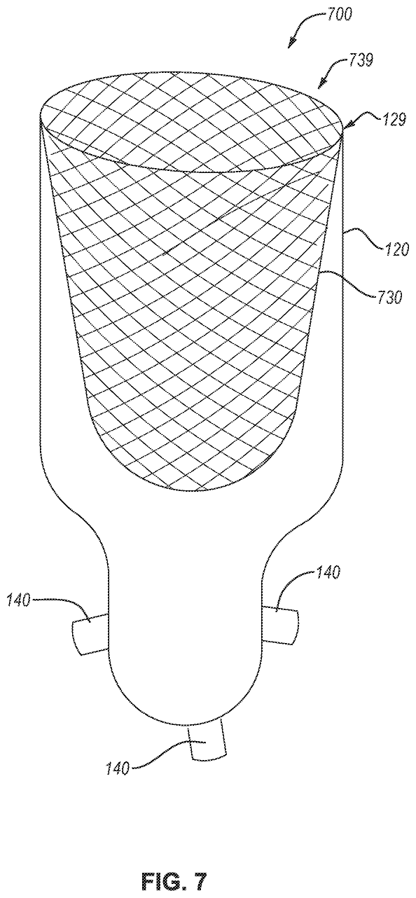

FIG. 7 is an isometric view of a partial assembly of a bodily waste collection device 700, according to an embodiment. The bodily waste collection device 700 is similar or identical to the bodily waste collection device 100 in one or more aspects. For example, the bodily waste collection device 700 includes the fluid impermeable barrier 120, the filter bag 730, and the one or more ports 140. The filter bag 730 is similar or identical to the filter bag 130 in one or more aspects. As shown, the filter bag 730 includes a mouth 739 at an upper end thereof. The mouth 739 of the filter bag 730 may be at least as wide as the mouth of the 129 of the fluid impermeable barrier 120. Accordingly, the mouth 739 of the filter bag 730 may be affixed to the fluid impermeable barrier 120, such as at the mouth 129 of the fluid impermeable barrier 120, as shown. The mouth of the filter bag 730 may be bound to the fluid impermeable barrier 120 by adhesive (e.g., any adhesive), stitching, heat welding, staples, integral formation, or the like. The fluid impermeable barrier 120 includes an upper portion having a greater outer dimension than a lower portion thereof. The filter bag 730 includes the one or more ports 140 in the lower portion or region thereof.

In some examples, the filter bag 730 may be affixed to the fluid impermeable barrier 120 along the interior surface thereof, such as around the inner diameter of the fluid impermeable barrier at an intermediate point between the mouth 129 and the lowermost extent of the fluid impermeable barrier 120. For example, the filter bag 730 may be spaced from the annular body (not shown) and mouth 129 of the fluid impermeable barrier 120 by a section of the interior surface of the fluid impermeable barrier 120.

Returning to FIG. 1, the bodily waste collection device 100 includes the one or more ports 140. The one or more ports 140 provide a passageway to fluidly connect the chamber 126 with one or more of the drainage tubing 150, a vacuum source, or fluid storage container. The one or more ports 140 are disposed on the fluid impermeable barrier 120. The one or more ports 140 may be positioned in locations at or near where liquid is expected to collect in the chamber 126. For example, the one or more ports 140 may be positioned on the fluid impermeable barrier 120 in locations expected to be at or near a gravimetrically low point of the fluid impermeable barrier 120 during use, such as when a wearer is sitting, laying, reclined, standing, or in any other position. The one or more ports 140 may be positioned in the lower portion of the fluid impermeable barrier 120 at the sump 128. Any number of ports may be used. For example, at least a single port 140 may be disposed in the fluid impermeable barrier 120.

The one or more ports 140 are sized and shaped to connect to drainage tubing 150 (e.g., drainage tube) to remove fluids collected in the chamber 126. For example, the one or more ports 140 are in fluid communication with the chamber 126 and are used to place the drainage tubing 150 in fluid communication with the chamber 126, such as to remove fluids therefrom. The one or more ports 140 may be sized and shaped to receive the drainage tubing 150 therein, such as being a female connection. The female connection may be sized and shaped to allow the drainage tubing 150 to insert therein. The one or more ports 140 may be configured to receive the drainage tubing 150 thereon, such as being a male connection (e.g., nipple or tube connection). When the drainage tubing 150 is connected to the one or more ports 140, the drainage tubing 150 fluidly couples the chamber 126 or sump 128 to one or more of a storage container or vacuum source via the one or more ports 140.

The drainage tubing 150 may include medical tubing. For example, the drainage tubing 150 may be constructed of one or more polymers such as silicone, latex, ethylene vinyl acetate (EVA), polytetrafluoroethylene (PTFE), silicone polyurethane, polyamide, polyurethane, polyethylene, other thermoplastics and block copolymers thereof, or any other suitable polymers for medical use. The fluids may be drained from the chamber via the one or more ports 140 fluidly coupled to the drainage tubing 150, such as via gravity, a vacuum force, or any other force.

As shown in FIG. 1, the bodily waste collection device 100 may include at least one input port 160. The at least one input port 160 may be similar or identical to the at least one port 140 in one or more aspects. The at least one input port 160 may be sized and shaped to connect to an input tube 170. The input tube 170 may be similar or identical to the drainage tubing 150 in one or more aspects. The input tube 170 may be operably coupled to in input source such as an air or liquid pump. The input tube 170 may input air into the chamber 126 such as to prevent suction from the ports 140 from reaching the wearer. The input tube 170 may be used to input a liquid into the chamber, such as water to flush out the chamber 126. The input port 160 may be positioned nearer the annular body 110 than the one or more ports 140, such as to allow the input material to be input between the suction applied at the one or more ports 140 and the wearer.

In some examples (not shown), the input port 160 and input tube 170 may be omitted. In such examples, the bodily waste collection device 100 may include one or more vents or one-way valves on the fluid impermeable barrier 120. Such one-way valves may be sized and shaped to open inwardly toward the chamber 126 responsive to a vacuum force applied thereto. Accordingly, vacuum applied in the chamber 126 may be prevented from reaching the wearer.

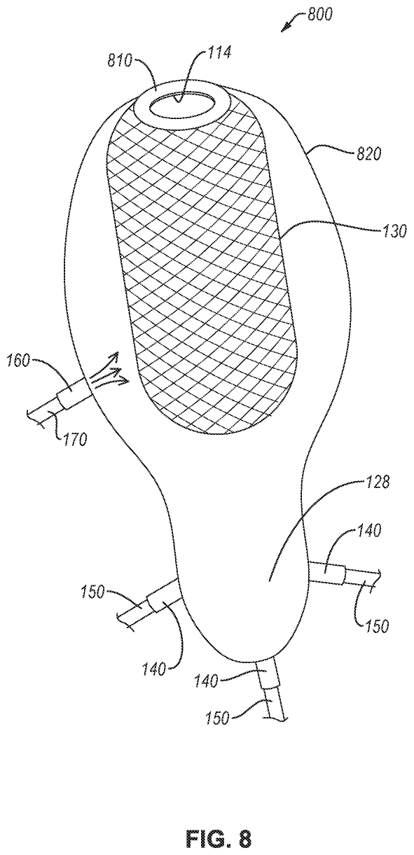

FIG. 8 is an isometric view of a bodily waste collection device 800, according to an embodiment. The bodily waste collection device 800 may be similar or identical to the bodily waste collection device 100, in one or more aspects. For example, the bodily waste collection device 800 includes the annular body 810, the fluid impermeable barrier 820, the filter bag 130, one or more ports 140, the drainage tubing 150, at least one input port 160, and the input tube 170.

The annular body 810 may be similar or identical to the annular body 110 (FIG. 1) in one or more aspects. As shown, the annular body 810 has smaller outer dimensions than the annular body 110. Accordingly, the annular bodies disclosed herein may have any outer dimensions, such as a smaller outer dimension that is selected to fit within the intergluteal cleft of a wearer during use. In such examples, the annular body 810 may not extend out of the intergluteal cleft of the wearer when worn.

The fluid impermeable barrier 820 may be similar or identical to the fluid impermeable barrier 120 in one or more aspects. As shown, the fluid impermeable barrier 820 may have a different shape or conformation than the fluid impermeable barrier 120 (FIG. 1). For example, and as shown in FIG. 8, the fluid impermeable barrier 820 may have a wider central portion than an upper portion and lower portion. The fluid impermeable barrier 820 may form the sump 128 within the chamber 126, such a lowermost portion of the fluid impermeable barrier 820. Such sump 128 may be a pocket formed in the fluid impermeable barrier 820 as shown.

As shown, the fluid impermeable barrier 820 may be at least partially inflated by positive pressure (e.g., air) applied via the input port 160 and input tube 170. The positive pressure may be provided to prevent the fluid impermeable barrier 820 from collapsing under vacuum applied via the one or more ports 140 and drainage tubing 150. The positive pressure may prevent the suction (applied via the vacuum and one or more ports) from reaching the anus or stoma or the wearer during use. The positive pressure may provide some spatial separation between the inner surface of the fluid impermeable barrier 820 and the filter bag 130 during use.

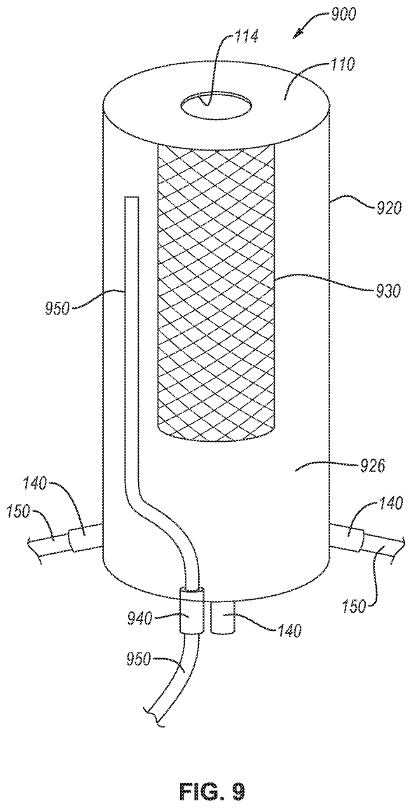

In some examples, the drainage tubing may extend through the input port(s) into the chamber of the bodily waste collection device. FIG. 9 is an isometric view of a bodily waste collection device 900, according to an embodiment. The bodily waste collection device 900 may be similar or identical to any of the bodily waste collection devices disclosed herein, in one or more aspects. The bodily waste collection device 900 includes the annular body 110, the fluid impermeable barrier 920, the filter bag 930, one or more ports 140 and 940, and the drainage tubing 150 and 950.

The annular body 110 is affixed to the fluid impermeable barrier 920. The fluid impermeable barrier 920 may be similar or identical to any of the fluid impermeable barriers disclosed herein (e.g., 120), in one or more aspects. The fluid impermeable barrier 920 may be sized and shaped as a pouch as shown. The inner surface of the fluid impermeable barrier 920 defines the chamber 926. The chamber 926 receives and retains the bodily waste therein.

The filter bag 930 is disposed within the chamber 926 and in fluid communication with the opening 114 of the annular body 110 to receive the waste that passes therethrough. The filter bag 930 may be similar or identical to any of the filter bags disclosed herein, in one or more aspects. For example, the filter bag 930 may have a longitudinal length and lateral dimensions that are smaller than the longitudinal length and lateral dimensions the fluid impermeable barrier 920. Accordingly, the fluid from the bodily waste received into the device 900 may pass through the filter bag 930 and settle at a gravimetrically low point within the chamber 926 or at a point of lowest pressure, such as proximate to the one or more ports 140.

The bodily waste collection device 900 includes the one or more additional ports 940 and drainage tubing 950. The one or more additional ports 940 and drainage tubing 950 may be similar or identical to the one or more ports 140 and drainage tubing 150 disclosed herein, in one or more aspects. For example, the additional port(s) 940 may be sized and shaped to receive the drainage tubing 950 disclosed therein. The drainage tubing 950 may pass through the port 940 and extend into the chamber 926, such as to position the distal end thereof in an upper region of the chamber 926. The distal end (e.g., open end) of the drainage tubing 950 may be disposed at any location within the chamber 926. The additional port(s) 940 and drainage tubing 950 may be used to remove fluids from the chamber 926 from additional locations spaced from the one or more ports 140. While shown as one additional port 940 and drainage tubing 950, it should be understood that multiple additional ports 940 and drainage tubing 950 may be utilized.

In some examples, the additional port 940 may be configured as an input port 160 (FIG. 1) and the drainage tubing 950 may be configured as an input tube 170. In such examples, the input tube 170 may be placed in any selected position within the chamber 926. Such positioning may be proximate to the filter bag to apply a liquid thereto to wash the contents of the filter bag.

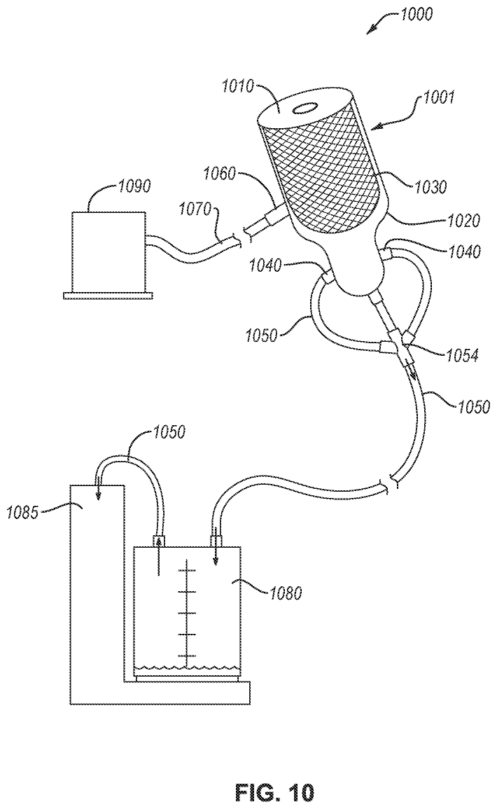

The bodily waste collection devices disclosed herein may be used in a system for collecting bodily waste. FIG. 10 is a schematic of a bodily waste collection system 1000, according to an embodiment. The system 1000 includes a bodily waste collection device 1001, a fluid storage container 1080, a vacuum source 1085, and input source 1090. The bodily waste collection device 1001 may be fluidly coupled to one or more of the fluid storage container 1080 or the vacuum source 1085 via the drainage tubing 1050. The bodily waste collection device 1001 may be fluidly coupled to the input source 1090 via the input tube 1070.

The bodily waste collection device 1001 may be similar or identical to any of the bodily waste collection devices disclosed herein, in one or more aspects. For example, the bodily waste collection device 1001 may include an annular body 1010, a fluid impermeable barrier 1020, a filter bag 1030, one or more ports 1040, and an input port 1060. The annular body 1010 be similar or identical to any of the annular bodies disclosed herein. The fluid impermeable barrier 1020 be similar or identical to any of the fluid impermeable barriers disclosed herein. The filter bag 1030 be similar or identical to any of the filter bags disclosed herein. The one or more ports 1040 be similar or identical to any of the ports disclosed herein. The input port 1060 be similar or identical to any of the input ports disclosed herein.

The drainage tubing 1050 may be fluidly coupled to of the one or more ports 140 and the fluid storage container 1080. Drainage tubing 1050 may be similar or identical to any of the drainage tubing disclosed herein, in one or more aspects. The fluids (e.g., liquid from bodily waste) may be removed from the chamber of the bodily waste collection device 1001 via the drainage tubing 150 and one or more ports 140. The fluid may travel through the drainage tubing 150 to the fluid storage container 1080, such as via vacuum force or gravity. The drainage tubing 1050 from a plurality of ports 1040 may be joined into single piece of drainage tubing 1050 via a manifold 1054. The manifold 1054 may include male or female connections for mating to the drainage tubing 1050.

Fluid storage container 1080 may include a bag (e.g., drainage bag), a bottle or cup (e.g., collection jar), or any other enclosed container for storing bodily fluids. The fluid storage container 1080 may be fluidly coupled to the vacuum source 1085, such as via a portion of drainage tubing 1050. The vacuum source 1085 may provide a vacuum for pulling fluids from the bodily waste collection device 1001 into the fluid storage container 1080. The fluid collected from the bodily waste collection device 1001 is moved through the drainage tubing 1050 into the fluid storage container 1080. By having a separate connection to the vacuum source 1085 on the fluid storage container 1080, the fluids removed from the bodily waste collection device 1001 may be prevented from entering the vacuum source 1085.

The vacuum source 1085 may include one or more of a manual vacuum pump, an electric vacuum pump, a diaphragm pump, a centrifugal pump, a displacement pump, a magnetically driven pump, a peristaltic pump, or any pump configured to produce a vacuum. The vacuum source 1085 may include a wall mounted suction line, such as found in a hospital room. The vacuum source 1085 provides a vacuum or suction to remove fluid from the bodily waste collection device 1001. In examples, the vacuum source 1085 may be powered by one or more of a power cord (e.g., connected to a power socket), one or more batteries, or even manual power (e.g., a hand operated vacuum pump). The vacuum source 1085 may include one or more of a switch, a button, a plug, a remote, or any other actuator suitable to activate the vacuum source 1085. The vacuum source 1085 may be selectively operated by a user (e.g., medical personnel, the wearer, or a caretaker).

The bodily waste collection device 1001 may be operably coupled to the input source 1090 via the input port 1060 and the input tube 1070. The input port 1060 and input tube 1070 may be similar or identical to any of the input ports and input tubes disclosed herein.

The input source 1090 may be used to input one or more fluids into the chamber of the bodily waste collection device 1001. The input source 1090 may include one or more of an air pump, a compressed air line, an oxygen source (e.g., compressed oxygen), a nitrogen source (e.g., compressed nitrogen), or any other source of gas. The input source 1090 may include one or more of a liquid source such as a water line, water pump, a saline pump, or the like. The input source 1090 may be used to selectively input a gas (e.g., air) into the chamber of the bodily waste collection device 1001 to prevent suction applied therein from collapsing the chamber or reaching the wearers skin, anus, or stoma. The input source 1090 may be used to selectively input a liquid (e.g., saline) into the chamber of the bodily waste collection device 1001 to clean the chamber or flush the chamber.

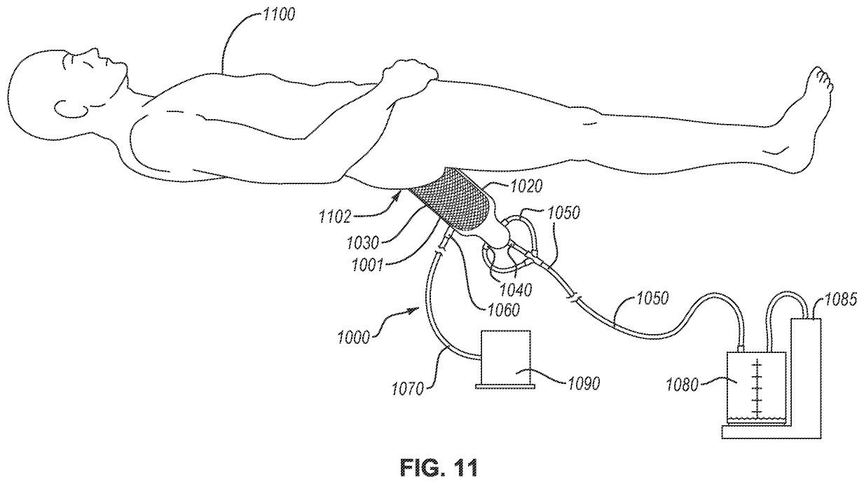

FIG. 11 is a schematic of the bodily waste collection system 1000 during use, according to an embodiment. The bodily waste collection device 1001 may be positioned on the wearer 1100, such as within the gluteal cleft 1102 of the wearer 1100. In such examples, the annular body of the bodily waste collection device 1001 may be positioned around the anus of the wearer 1100 such that the opening (not shown) of the annular body (not shown) is disposed over the anus of the wearer 1100. At least a portion of the bodily waste collection device 1001 may be retained within the gluteal cleft 1102 of the wearer 1100. For example, the annular body may be adhered to the skin of the wearer 1100 around the anus and when the wearer's legs are closed, the annular body will be deformed (e.g., folded) in the gluteal cleft 1102.

As the wearer 1100 has bowel movements, the waste (e.g., stool) is collected in the filter bag 1030 via the opening. The solids are retained in the filter bag 1030 while liquid drains from the waste and passes out of the filter bag 1030 into the chamber of the bodily waste collection device 1001. Such drainage may be accomplished by one or more of gravity or a vacuum applied in the chamber by the vacuum source 1085. The liquid is removed from the chamber via the one or more ports and the drainage tubing 1050. The liquid is deposited in the fluid storage container 1080.

Optionally, the input source 1090 may input one or more fluids into the bodily waste collection device 1001 via the input port(s) 1060 and the input tube 1070. For example, a stream of air may be passed into the chamber from the input source 1090 while the vacuum is applied therein from the vacuum source 1085 to prevent the chamber from collapsing or the suction from reaching the wearer's tissue (e.g., skin). A liquid may be input into the chamber from the input source as disclosed herein.

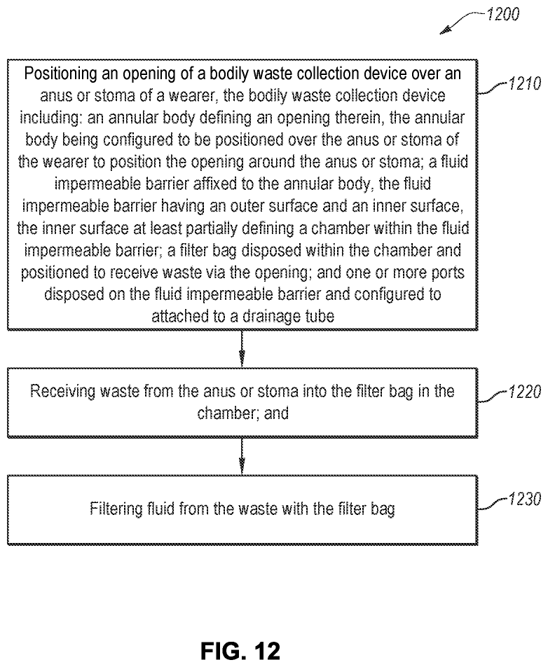

FIG. 12 is a flow diagram of a method 1200 to collect bodily waste, according to an embodiment. The method 1200 includes block 1210, which recites “positioning an opening of a bodily waste collection device over an anus or stoma of a wearer, the bodily waste collection device including an annular body defining an opening therein, the annular body being configured to be positioned over the anus or stoma of the wearer to position the opening around the anus or stoma, a fluid impermeable barrier affixed to the annular body, the fluid impermeable barrier having an outer surface and an inner surface, the inner surface at least partially defining a chamber within the fluid impermeable barrier; a filter bag disposed within the chamber and positioned to receive waste via the opening; and one or more ports disposed on the fluid impermeable barrier and configured to attached to a drainage tube.” Block 1210 may be followed by block 1220, which recites “receiving waste from the anus or stoma into the filter bag in the chamber.” Block 1220 may be followed by block 1230, which recites “filtering fluid from the waste with the filter bag.” Blocks 1210, 1220, 1230 of the method 1200 are for illustrative purposes. For example, the blocks may be modified, supplemented, split, or combined. In an example, one or more of the blocks 1210, 1220, 1230 of the method 1200 may be omitted from the method 1200. Any of the blocks 1210, 1220, or 1230 may include using any of the bodily waste collection devices or systems disclosed herein.

Block 1210 recites “positioning an opening of a bodily waste collection device over an anus or stoma of a wearer, the bodily waste collection device including an annular body defining an opening therein, the annular body being configured to be positioned over the anus or stoma of the wearer to position the opening around the anus or stoma, a fluid impermeable barrier affixed to the annular body, the fluid impermeable barrier having an outer surface and an inner surface, the inner surface at least partially defining a chamber within the fluid impermeable barrier; a filter bag disposed within the chamber and positioned to receive waste via the opening; and one or more ports disposed on the fluid impermeable barrier and configured to attached to a drainage tube.” The bodily waste collection device or components thereof may be similar or identical to any of the bodily waste collection devices disclosed herein, in one or more aspects.

Positioning the opening of the bodily waste collection device over the anus or stoma of a wearer may include positioning the opening over the anus or stoma such that the opening at least partially (e.g., completely) encircles the anus or stoma. Accordingly, bodily waste passed through the anus or stoma may pass through the opening into the bodily waste collection device. Positioning the opening of the bodily waste collection device over the anus or stoma of a wearer may include adhering the annular body to the wearer, such as in the intergluteal cleft of the wearer. Positioning the opening of the bodily waste collection device over the anus or stoma of a wearer may include deforming a conformation of the annular body to comply with the anatomy of the wearer (e.g., surfaces of one or more anatomical features) around the anus or stoma, such as by one or more of bending or folding. The wearer or a user (e.g., caretaker, medical professional, etc.) may position the opening of the bodily waste collection device over the anus or stoma of a wearer

Block 1220 recites “receiving waste from the anus or stoma into the filter bag in the chamber.” For example, receiving waste from the anus or stoma into the filter bag in the chamber includes receiving the waste into the filter bag via the opening.

Block 1230 recites, “filtering fluid from the waste with the filter bag.” In some examples, filtering fluid from the waste with the filter bag includes utilizing gravity to sieve the fluid from the waste. In some examples, filtering fluid from the waste with the filter bag includes utilizing a vacuum to separate the fluid from solids in the waste. The vacuum may be applied from the vacuum source(s) disclosed herein, such as via the one or more ports and drainage tube (e.g., drainage tubing). The bodily waste collection device may be a portion of a system for collecting bodily waste, such as any of the systems disclosed herein. For example, the bodily waste collection device is fluidly coupled to a vacuum source via the drainage tube. In such examples, the method 1200 includes applying suction with the vacuum source via the one or more ports remove the fluid from the chamber.

Filtering fluid from the waste with the filter bag may include retaining solids from the waste in the filter bag. Accordingly, semi-solid bodily waste may be filtered in the filter bag to retain the solids therein.

The method 1200 may include removing the fluid from the chamber. Removing fluid from the chamber may include removing the fluid via the one or more ports and drainage tubing or tube, such as via gravity or vacuum. The vacuum may be selectively applied using the vacuum source. For example, the wearer or a user (e.g., medical professional) may visually identity liquid pooling in the chamber or stool in the filter bag through the transparent fluid impermeable barrier, and responsive thereto, activate the vacuum source to remove the liquid from the chamber and/or bodily waste.

In some examples, the method 1200 may include collecting the fluid(s) that are removed from the bodily waste collection device, such as into a fluid storage container that is spaced from the bodily waste collection device and fluidly coupled to the drainage tubing. The fluid storage container may include any of the fluid storage containers disclosed herein. The collected fluid may be quantified or analyzed.

The method 1200 may include inputting a fluid into the chamber. Inputting fluid into the chamber may include utilizing any of the input ports, input tubes, and input sources disclosed herein, such as a gas source or liquid source. Inputting fluid into the chamber may include inputting a gas into the chamber, such as air, oxygen, nitrogen, or the like. Inputting gas into the chamber may be carried out to prevent vacuum applied into the chamber from reaching the tissue of the wearer. For example, inputting the gas into the chamber may be carried out contemporaneously with (e.g., simultaneously or close in time to) applying a vacuum in the chamber. The input port may be disposed at a point in the fluid impermeable barrier nearer the wearer than the one or more drainage ports. Accordingly, the suction from the drainage ports may be prevented from reaching the wearer by inputting gas into the chamber nearer the wearer. Inputting gas into the chamber may be carried out to increase the volume of the chamber.

Inputting a fluid into the chamber may include inputting liquid into the chamber, such as water, saline, a cleaning fluid, or the like. Inputting liquid into the chamber may include inputting a saline or salt solution into the chamber to clean or flush at least a portion of the chamber.

In some examples, the method 1200 may include changing the fluid collection device for a new fluid collection device, such as when the filter bag fills up. Such a change may be responsive to visually confirming that the filter bag is filled to a point where a change of devices is desired, such as confirming via viewing the filter bag through the transparent fluid impermeable barrier.

While the devices, systems, and methods disclosed herein can be used over a stoma, the devices, systems, and methods disclosed herein may be used to prevent the necessity of ostomy and stoma for removing stool from a patient. As an alternative to an ostomy, the bodily waste collection devices disclosed herein may be used to non-intrusively and hygenically receive bodily waste from patients. Additionally, the useful life of the devices may be lengthened by removing the fluids from the bodily waste received therein, which is possible by retaining the solids from the waste within the filter bag.

While various aspects and embodiments have been disclosed herein, other aspects and embodiments are contemplated. The various aspects and embodiment disclosed herein are for purposes of illustration and are not intended to be limiting. Features from any of the disclosed embodiments may be used in combination with one another, without limitation.

What is claimed is:

1. A bodily waste collection device, comprising:
an annular body defining an opening therein, the annular body being configured to be positioned over an anus or a stoma of a wearer to position the opening around the anus or the stoma;
a fluid impermeable barrier affixed to the annular body, the fluid impermeable barrier having an outer surface and an inner surface, the inner surface at least partially defining a chamber within the fluid impermeable barrier;
a filter bag disposed within the chamber and positioned to receive waste via the opening; and
one or more ports disposed on the fluid impermeable barrier and configured to attach to a drainage tube.

2. The bodily waste collection device of claim 1 wherein the annular body includes an annular plate sized and shaped to fit within an intergluteal cleft and having an adhesive disposed on an outward facing surface at least partially around the opening.

3. The bodily waste collection device of any of claims 1-2 wherein the fluid impermeable barrier includes an upper portion having a greater outer dimension than a lower portion of the fluid impermeable barrier and the one or more ports are disposed in the lower portion.

4. The bodily waste collection device of any of claims 1-3 wherein the fluid impermeable barrier forms a sump in a portion of the chamber.

5. The bodily waste collection device of any of claims 1-4 wherein the filter bag includes a mesh bag having a sieve size selected to allow fluids to pass therethrough and retain solids therein.

6. The bodily waste collection device of any of claims 1-5 wherein the filter bag is affixed to the annular body at least proximate to the opening.

7. The bodily waste collection device of any of claims 1-6 wherein the filter bag is affixed to an underside of the annular body between the opening and the fluid impermeable barrier.

8. The bodily waste collection device of any of claims 1-7 wherein the filter bag is affixed to the fluid impermeable barrier.

9. The bodily waste collection device of any of claims 1-8 wherein the filter bag has a length that is shorter than a length of the fluid impermeable barrier.

10. The bodily waste collection device of any of claims 1-9 wherein the one or more ports are disposed in a lower region of the fluid impermeable barrier.

11. The bodily waste collection device of any of claims 1-10 wherein the one or more ports are sized to receive the drainage tube therein.

12. A bodily waste collection system, comprising:
a fluid storage container configured to hold a fluid;
a bodily waste collection device fluidly coupled to the fluid storage container, the bodily waste collection device including:
an annular body defining an opening therein, the annular body being configured to be positioned over an anus or a stoma of a wearer to position the opening around the anus or the stoma;
a fluid impermeable barrier affixed to the annular body, the fluid impermeable barrier having an outer surface and an inner surface, the inner surface at least partially defining a chamber within the fluid impermeable barrier;
a filter bag disposed within the chamber and positioned to receive waste via the opening; and
one or more ports disposed on the fluid impermeable barrier and configured to attached to a drainage tube; and
a drainage tube in fluid communication with the one or more ports; and
a vacuum source fluidly coupled to one or more of the fluid storage container or the bodily waste collection device via the drainage tube, the vacuum source configured to draw fluid from the chamber via the drainage tube.

13. The bodily waste collection system of claim 12 wherein the annular body is sized and shaped to fit within an intergluteal cleft of the wearer and includes an adhesive disposed on an outward facing surface at least partially around the opening.

14. The bodily waste collection system of any of claims 12-13 wherein the filter bag includes a mesh bag having a sieve size selected to allow fluids to pass therethrough and retain solids therein.

15. The bodily waste collection system of any of claims 12-14 wherein the one or more ports are disposed in a lower region of the fluid impermeable barrier.

16. The bodily waste collection system of any of claims 12-15 wherein the drainage tube is attached to a plurality of the one or more ports by a manifold and separate drainage tubes.

17. The bodily waste collection system of any of claims 12-16 wherein the vacuum source is fluidly coupled to the fluid storage container and the vacuum source is configured to produce a vacuum in the chamber to draw fluid from the chamber into the fluid storage container via the drainage tube.

18. The bodily waste collection system of any of claims 12-17, further comprising:
at least one input port located in an upper portion of the fluid impermeable barrier; and
an air pump in fluid communication with the input port via an input tube therebetween, wherein the air pump is configured to input air into the chamber via the input tube.

19. A method to collect bodily waste, the method comprising:
positioning an opening of a bodily waste collection device over an anus or a stoma of a wearer, the bodily waste collection device including:
an annular body defining an opening therein, the annular body being configured to be positioned over the anus or stoma of the wearer to position the opening around the anus or the stoma;
a fluid impermeable barrier affixed to the annular body, the fluid impermeable barrier having an outer surface and an inner surface, the inner surface at least partially defining a chamber within the fluid impermeable barrier;
a filter bag disposed within the chamber and positioned to receive waste via the opening; and
one or more ports disposed on the fluid impermeable barrier and configured to attached to a drainage tube;
receiving waste from the anus or stoma into the filter bag in the chamber; and
filtering fluid from the waste with the filter bag.

20. The method of claim 19 wherein positioning the opening of the bodily waste collection device over the anus or stoma of a wearer includes adhering the annular body to the wearer in an intergluteal cleft of the wearer.

21. The method of any of claims 19-20 wherein positioning the opening of the bodily waste collection device over the anus or stoma of a wearer includes deforming a conformation of the annular body to comply with the anatomy of the wearer around the anus or stoma.

22. The method of any of claims 19-21 wherein receiving waste from the anus or stoma into the filter bag in the chamber includes receiving the waste into the filter bag via the opening.

23. The method of any of claims 19-22 wherein filtering fluid from the waste with the filter bag includes utilizing gravity to sieve the fluid from the waste.

24. The method of any of claims 19-23 wherein filtering fluid from the waste with the filter bag includes utilizing a vacuum to separate the fluid from solids in the waste.

25. The method of any of claims 19-24 further comprising removing the fluid from the chamber.

26. The method of any of claims 19-25 wherein the bodily waste collection device is fluidly coupled to a vacuum source via the drainage tube.

27. The method of claim 26, further comprising applying suction with the vacuum source via the one or more ports remove the fluid from the chamber.
